(19) United States
(12) Patent Application Publication
(10) Pub. No.: US 2025/0152264 A1
(43) Pub. Date: May 15, 2025

(54) COUPLING DEVICES AND RELATED SYSTEMS

(71) Applicant: Neuwave Medical, Inc., Madison, WI (US)

(72) Inventors: Louis MINGIONE, Madison, WI (US); Mark THOM, Madison, WI (US); Jeff BISSING, Madison, WI (US); David JAEKEL, Redwood City, CA (US)

(21) Appl. No.: 18/729,613

(22) PCT Filed: Feb. 17, 2023
(86) PCT No.: PCT/IB2023/051461

§ 371 Date: Jul. 17, 2024

Related U.S. Application Data
(60) Provisional application No. 63/311,762, filed on Feb. 18, 2022.

(51) Int. Cl. A61B 34/30; A61B 18/00

(52) U.S. Cl. CPC A61B 34/30; A61B 18/00; A61B 2018/00982; A61B 2034/301

(57) ABSTRACT

The present invention relates to a coupling device capable of simultaneously a) attaching onto a robotically controlled endoscope system, and b) securing a cable connected with a generator (e.g., capable of providing microwave energy) and a medical procedure device (e.g., microwave energy delivery device), and related systems and methods.

### FIELD OF INVENTION

The present invention relates to a coupling device capable of simultaneously a) attaching onto a robotically controlled endoscope system, and b) securing a cable connected with a generator (e.g., capable of providing microwave energy) and a medical procedure device (e.g., microwave energy delivery device), and related systems and methods.

### BACKGROUND

Medical procedures involving delivery of ablation energy with robotically controlled endoscope systems requires the organization of several items (e.g., energy generators, microwave ablation devices, robotically controlled endoscope systems) and related accessory cords. Organization of such items and related accessory cords can be challenging. Disorganization of such items can compromise the intended result of the medical procedure.

The present invention addresses this problem.

### SUMMARY

Ablation procedures involving robotically controlled endoscope systems require careful organization with microwave ablation energy delivery devices, microwave energy generators, and accessory cables. Optimization of such medical procedures requires positioning of robotically controlled endoscope arms, the microwave ablation energy delivery devices, and the accessory cable connecting with the generator and the microwave ablation energy delivery devices to be within close proximity. Ensuring such close proximity can be cumbersome. The present invention provides coupling devices capable of solving these problems.

Indeed, the present invention relates to a coupling device capable of simultaneously a) attaching onto a robotically controlled endoscope system, and b) securing a cable connected with a generator (e.g., capable of providing microwave energy) and a medical procedure device (e.g., microwave energy delivery device), and related systems and methods.

In certain embodiments, the present invention provides coupling devices configured for attachment onto an endoscope system, and configured for securing a cable, wherein the cable has a cable proximal end and a cable distal end, wherein the coupling device is configured to secure the cable distal end; wherein the coupling device is configured to attach onto the endoscope system and secure the cable distal end such that the coupling device, the endoscope system, and the cable distal end are within close proximity.

The coupling devices are not limited to a certain close proximity for the coupling device, the endoscope system, and the cable distal end are within close proximity. In some embodiments, the close proximity is within a 24 inch or smaller area (e.g., 12 inch or smaller area; 6 inch or smaller area; 3 inch or smaller area; 1 inch or smaller area).

In some embodiments, the coupling device is configured for attachment onto the endoscope system such that the coupling device is immobilized in relation to the endoscope system. In some embodiments, the coupling device is configured to secure the cable distal end such that the cable distal end is immobilized in relation to the coupling device.

In some embodiments, the coupling device is configured to secure the cable distal end in a manner permitting the cable distal end to securely connect with a proximal end of a medical procedure device such that one or more materials can be provided to and/or received from the medical procedure device, wherein the cable proximal end is configured to connect with a generator such that the generator can provide one or more materials to the cable proximal end, wherein the one or more materials is microwave energy, radiofrequency energy, fluid, temperature obtaining agents, and imaging obtaining agents. In some embodiments, the fluid is a pressurized gas or liquid. In some embodiments, the fluid is carbon dioxide. In some embodiments, the fluid is water.

In some embodiments, the medical procedure device is selected from a microwave energy delivery device, a radiofrequency energy delivery device, a cryo energy delivery device, an imaging device, temperature monitoring device, and a temperature regulating device. In some embodiments, the microwave energy delivery device comprises a coaxial design or a triaxial design. In some embodiments, the energy delivery device is configured to circulate coolant received from the cable.

In some embodiments, the endoscope system is a robotically controlled endoscope system wherein the robotically controlled endoscope system comprises one or more robotically controlled endoscope system arms configured to assist in manipulation of a medical procedure device connected with the cable distal end secured in the coupling device, wherein each robotically controlled endoscope system arm has a robotically controlled endoscope system arm proximal end, a robotically controlled endoscope system arm distal end, a robotically controlled endoscope system arm main body.

In some embodiments, the coupling device is configured to attach onto one of the robotically controlled endoscope system arms such that the coupling device is immobilized with respect to the robotically controlled endoscope system arm. In some embodiments, the coupling device is configured to attach onto one of the robotically controlled endoscope system arm distal ends such that the coupling device is immobilized with respect to the robotically controlled endoscope system arm distal end. In some embodiments, the coupling device is configured to attach onto one of the robotically controlled endoscope system arms via one or more of: a magnet-based attachment, a shape-locking attachment, a suction-based attachment, a Velcro® attachment, and an adhesive attachment.

In some embodiments, the coupling device has an open-hollow shape, has a coupling device interior surface, wherein the coupling device interior surface is configured to contact the robotically controlled endoscope system arm distal end, wherein the coupling device has a coupling device exterior surface, wherein the coupling device exterior surface has a coupling device exterior surface attachment region configured to secure the cable distal end such that the cable distal end is immobilized with respect to the coupling device.

In some embodiments, the open-hollow shape of the coupling device is configured to match at least a portion of the shape of the robotically controlled endoscope system arm distal end such that the coupling device is able to fit over and around the robotically controlled endoscope system arm distal end and attach onto the robotically controlled endoscope system arm distal end such that the coupling device is immobilized with respect to the robotically controlled endoscope system arm distal end.

In some embodiments, the coupling device is capable of attachment onto the robotically controlled endoscope system arm distal end by passing the robotically controlled endoscope system arm distal end through the open-hollow shape of the coupling device until the coupling device interior surface contacts the robotically controlled endoscope system arm distal end resulting immobilization of the coupling device with respect to the robotically controlled endoscope system arm distal end.

In some embodiments, the coupling device is configured, upon being secured onto the robotically controlled endoscope system arm distal end, to be tightened around the robotically controlled endoscope system arm distal end for purposes of further securing the coupling device with the relation to the robotically controlled endoscope system arm distal end. In some embodiments, the coupling device is configured, upon being secured onto the robotically controlled endoscope system arm distal end, to be loosened for purposes of releasing the coupling device from the robotically controlled endoscope system arm distal end.

In some embodiments, the coupling device has an open-hollow uniform ring shape or an open-hollow oval shape.

In some embodiments, the coupling device exterior surface attachment region is configured to secure the cable distal end such that the cable distal end is immobilized with respect to the coupling device via one or more of: a magnet-based attachment, a shape-locking attachment, a suction-based attachment, a Velcro® attachment, and an adhesive attachment.

In some embodiments, the coupling device comprises a coupling device first arm, wherein the coupling device first arm has a coupling device first arm proximal end, a coupling device first arm main body, and a coupling device first arm distal end.

In some embodiments, the coupling device comprises a coupling device second arm, wherein the coupling device second arm has a coupling device second arm proximal end, a coupling device second arm main body, and a coupling device second arm distal end.

In some embodiments, the coupling device first arm proximal end and the coupling device second arm proximal end are connected via a coupling device hinge, wherein the coupling device first arm and the coupling device second arm are each configured to linearly rotate about the coupling device hinge such that an expandable or retractable opening exists between the coupling device first arm proximal end and the coupling device second arm proximal end, wherein the coupling device first arm distal end is configured to lockingly connect with the coupling device second arm distal end such that the coupling device first arm, the coupling device second arm, and the coupling device hinge form a closed loop.

In some embodiments, the coupling device first arm and the coupling device second arm are configured to linearly rotate about the coupling device hinge such that an opening is generated between the coupling device first arm distal end and the coupling device second arm distal end, wherein the opening is of a sufficient size such that at least a portion of the robotically controlled endoscope system arm distal end can be positioned within the opening, wherein the coupling device first arm distal end is configured to lockingly connect with the coupling device second arm distal end while the robotically controlled endoscope system arm distal end is positioned within the opening thereby forming a closed loop around the robotically controlled endoscope system arm distal end in such a manner that the coupling device is immobilized with respect to the robotically controlled endoscope system arm distal end.

In some embodiments, the coupling device is configured, upon being secured onto the robotically controlled endoscope system arm distal end, to be tightened around the robotically controlled endoscope system arm distal end for purposes of further securing the coupling device with the relation to the robotically controlled endoscope system arm distal end, wherein the coupling device first arm distal end is configured to disconnect with the coupling device second arm distal end for purposes of releasing the coupling device from the robotically controlled endoscope system arm distal end.

In some embodiments, the coupling device first arm distal end has a coupling device first arm distal end opening configured to receive a screw, wherein the coupling device second arm distal end has a coupling device second arm distal end opening configured to receive a screw, wherein the coupling device is configured, upon being secured onto the robotically controlled endoscope system arm distal end, to be tightened around the robotically controlled endoscope system arm distal end for purposes of further securing the coupling device with the relation to the robotically controlled endoscope system arm distal end via a screw positioned within both the coupling device first arm distal end opening and the coupling device second arm distal end opening. In some embodiments, the screw is a thumb-screw.

In some embodiments, the coupling device exterior surface attachment region is positioned along the coupling device first arm main body, or the coupling device exterior surface attachment region is positioned along the coupling device second arm main body.

In some embodiments, the coupling device exterior surface attachment region is configured to secure the cable distal end such that the cable distal end is immobilized with respect to the coupling device via one or more of: a magnet-based attachment, a shape-locking attachment, a suction-based attachment, a Velcro® attachment, and an adhesive attachment.

In some embodiments, the coupling device has a plastic composition, a metal composition, a ceramic composition, or any mixture thereof.

In certain embodiments, the present invention provides systems comprising: a coupling device as described herein; a cable having a cable proximal end and a cable distal end; a medical procedure device; and a robotically controlled endoscope system, wherein the robotically controlled endoscope system comprises one or more robotically controlled endoscope system arms configured to assist in placement of or provide a conduit for a medical procedure device connected with the cable distal end secured in the coupling device, wherein each robotically controlled endoscope system arm has a robotically controlled endoscope system arm proximal end, a robotically controlled endoscope system arm distal end, a robotically controlled endoscope system arm main body.

In some embodiments, the medical procedure device is selected from a microwave energy delivery device, a radiofrequency energy delivery device, a cryo energy delivery device, an imaging device, temperature monitoring device, and a temperature regulating device. In some embodiments, the microwave energy delivery device comprises a coaxial design or a triaxial design.

In some embodiments, the system further comprises a generator configured to connect with the cable proximal end, wherein the generator is configured to provide one or more materials to the cable proximal end, wherein the one or more materials is microwave energy, radiofrequency energy, fluid, temperature obtaining agents, and imaging obtaining agents.

In certain embodiments, the present invention provides methods of conducting a medical procedure, comprising providing a system as described herein; securing the cable proximal end with a generator; securing the coupling device onto one of the robotically controlled endoscope system arm distal ends such that the coupling device is immobilized with respect to the robotically controlled endoscope system distal arm; securing the cable distal end with the coupling device attachment region such that the cable distal end is immobilized with respect to the coupling device; connecting the medical procedure device with the cable distal end; contacting a subject with the medical procedure device connected with the cable distal end; administering to the subject a medical procedure with the medical procedure device connected with the cable distal end.

In some embodiments, the medical procedure device is a microwave energy delivery device.

In some embodiments, the medical procedure is a microwave ablation procedure.

In some embodiments, the subject is a human subject.

In some embodiments, the generator is configured to provide one or more materials to the cable proximal end, wherein the one or more materials is microwave energy, radiofrequency energy, fluid, temperature obtaining agents, and imaging obtaining agents.

Additional embodiments are described herein.

### BRIEF DESCRIPTION OF THE DRAWINGS

FIG. 1-2 show a schematic of a coupling device.

FIGS. 3-5 show an exemplary embodiment of a coupling device.

FIGS. 6-7 shows an exemplary coupling device mated with a robotic arm.

FIGS. 8-9 show another exemplary embodiment of a coupling device.

FIGS. 10-11 shows coupling device embodied in FIGS. 8-9 mated with a robotic arm.

### DETAILED DESCRIPTION

The present invention relates to a coupling device capable of simultaneously a) attaching onto a robotically controlled endoscope system, and b) securing a cable connected with a generator (e.g., capable of providing microwave energy) and a medical procedure device (e.g., microwave energy delivery device), and related systems and methods.

The coupling devices of the present invention are not limited to particular size dimensions. Indeed, in some embodiments, the size dimension of the coupling device is such that it is able to attach onto an endoscope system (e.g., a robotically controlled endoscope system) and secure a cable connected with a medical procedure device without inhibiting the function of the endoscope system and the medical procedure device.

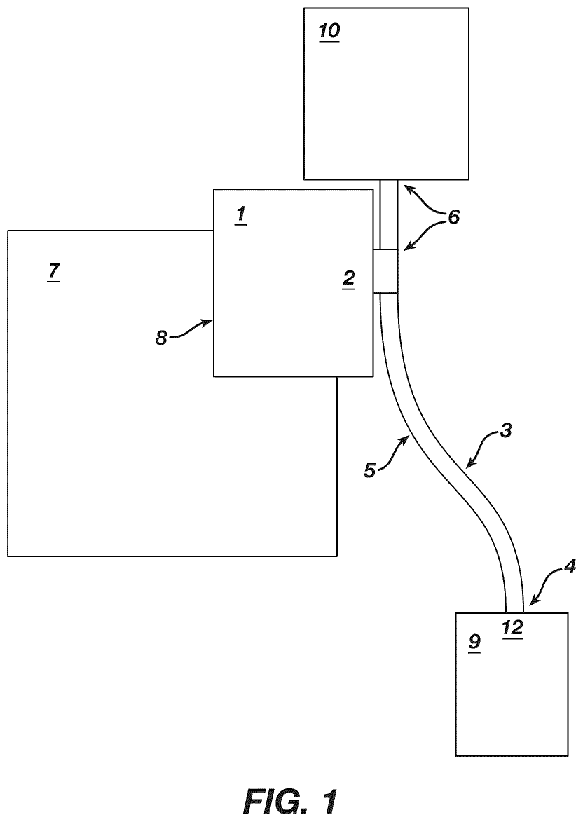

FIG. 1 shows a schematic of a coupling device 1 having a coupling device attachment region 2, a cable 3 having a cable proximal end 4, a cable main body 5, and a cable distal end 6, an endoscope system 7 having an endoscope system coupling device attachment region 8, a generator 9, a medical procedure device 10. As shown, the coupling device 1 is attached onto the endoscope system 7 at the endoscope system coupling device attachment region 8. As shown, the cable distal end 6 is secured with the coupling device 1 at the coupling device attachment region 2. As shown, the cable proximal end 4 is connected with the generator 9 at the generator cable proximal end connection region 12. As shown, the cable 3 is engaged with the medical procedure device 10 at the cable distal end 6.

Still referring to FIG. 1, the size dimension of the coupling device 1 is such that can attach onto an endoscope system 7 (e.g., a robotically controlled endoscope system) and secure a cable 3 connected with the generator 9 and a medical procedure device 10 such that the endoscope system 7, the medical procedure device 10, and the coupling device 1 are all within a close proximity. Such embodiments are not limited to a specific close proximity. In some embodiments, the coupling device 1 is capable of attaching onto an endoscope system 7 (e.g., a robotically controlled endoscope system) and securing a cable 3 connected with the endoscope system 7 and a medical procedure device 10 such that the coupling device 1, endoscope system 7, and cable 3 are within a 24 inch or smaller area (e.g., 20 inch or smaller area, 15 inch or smaller area, 10 inch or smaller area, 9 inch or smaller area, 5 inch or smaller area, 4 inch or smaller area, 3 inch or smaller area, 2 inch or smaller area, 1 inch or smaller area).

Still referring to FIG. 1, the coupling device 1 is not limited to a particular manner of attachment onto the endoscope system 7. In some embodiments, the coupling device 1 is configured to attach onto the endoscope system 7 at the endoscope system coupling device attachment region 8 such that the coupling device 1 is immobilized with respect to the endoscope system coupling device attachment region 8. The coupling device 1 is not limited to a particular manner of attachment onto the endoscope system coupling device attachment region 8. In some embodiments, the coupling device 1 attaches with the endoscope system coupling device attachment region 8 via one or more of: a magnet-based attachment, a shape-locking attachment, a suction-based attachment, a Velcro® attachment, and an adhesive attachment.

Still referring to FIG. 1, the coupling device 1 is not limited to a particular manner of securing the cable 3. In some embodiments, the coupling device 1 secures the cable distal end 6 such that cable distal end 6 is immobilized with respect to the coupling device 1.

Still referring to FIG. 1, the generator 9 is not limited to a particular manner of connection with the cable 3. In some embodiments, the generator 9 connects with the cable proximal end 4 at the generator cable proximal end connection region 12 such the cable proximal end 4 is immobilized with respect to the generator 9.

Still referring to FIG. 1, the medical procedure device 10 is not limited to a particular manner of connection with the cable 3. In some embodiments, the medical procedure device 10 is releasably connected with the cable distal end 6.

Still referring to FIG. 1, a simultaneous connection of 1) the cable proximal end 4 with the generator 9 and 2) the cable distal end 6 with the medical procedure device 10 permits the providing and retrieving of one or more materials between the generator 9 and the medical procedure device 10 via the cable 3. Such embodiments are not limited to particular types of materials capable of transfer to and from the generator 9 and the medical procedure device 10 via the cable 3. In some embodiments, the materials include, but are not limited to, microwave energy, radiofrequency energy, fluid, temperature obtaining agents (e.g., temperature sensors), and imaging obtaining agents (e.g., devices capable of obtaining image information).

Such embodiments are not limited to use of a specific type or kind of fluid. In some embodiments, the fluid is selected from water, glycol, air, inert gasses, carbon dioxide (CO2), nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, and a coolant gas compressed at or near its critical point.

Such embodiments are not limited to particular type or kind of generator 9. In some embodiments, the generator 9 is able to provide to the cable 3 one or more of: microwave energy, radiofrequency energy, fluid, temperature obtaining agents (e.g., temperature sensors), and imaging obtaining agents (e.g., devices capable of obtaining image information).

Such embodiments are not limited to a particular type or kind of medical procedure device 10. In some embodiments, the medical procedure device 10 is selected from an energy delivery device, a microwave energy delivery device, a cryo-energy delivery device, a radiofrequency energy delivery device, an imaging device, temperature monitoring device, and a temperature regulating device.

Such embodiments are not limited to a particular type or kind of energy delivery device (e.g., ablation device, surgical device, etc.) (see, e.g., U.S. Pat. Nos. 7,101,369, 7,033,352, 6,893,436, 6,878,147, 6,823,218, 6,817,999, 6,635,055, 6,471,696, 6,383,182, 6,312,427, 6,287,302, 6,277,113, 6,251,128, 6,245,062, 6,026,331, 6,016,811, 5,810,803, 5,800,494, 5,788,692, 5,405,346, 4,494,539, U.S. patents application Ser. Nos. 11/728,460, 11/728,457, 11/728,428, 11/237,136, 11/236,985, 10/980,699, 10/961,994, 10/961,761, 10/834,802, 10/370,179, 09/847,181; Great Britain Patent Application Nos. 2,406,521, 2,388,039; European Patent No. 1395190; and International Patent Application Nos. WO 06/008481, WO 06/002943, WO 05/034783, WO 04/112628, WO 04/033039, WO 04/026122, WO 03/088858, WO 03/039385 WO 95/04385; each herein incorporated by reference in their entireties). Such energy delivery devices are not limited to emitting a particular kind of energy. In some embodiments, the energy delivery devices are capable of emitting radiofrequency energy. In some embodiments, the energy delivery devices are capable of emitting microwave energy. Such devices include any and all medical, veterinary, and research applications devices configured for energy emission, as well as devices used in agricultural settings, manufacturing settings, mechanical settings, or any other application where energy is to be delivered.

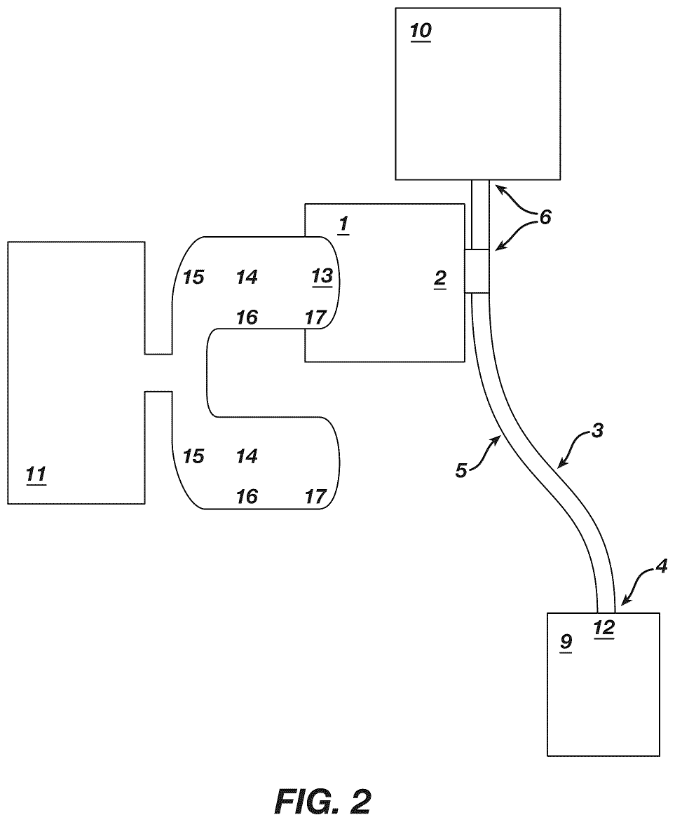

Such embodiments are not limited to a specific type or kind of endoscope system. In some embodiments, the endoscope system is a robotically controlled endoscope system. FIG. 2 shows a schematic of a coupling device 1 having a coupling device attachment region 2, a cable 3 having a cable proximal end 4, a cable main body 5, and a cable distal end 6, a generator 9, a medical procedure device 10, and a robotically controlled endoscope system 11 having a robotically controlled endoscope system coupling device attachment region 13. FIG. 2 further shows that the robotically controlled endoscope system 11 has robotically controlled endoscope system arms 14 with each having a robotically controlled endoscope system arm proximal end 15, a robotically controlled endoscope system arm main body 16, and a robotically controlled endoscope system distal end 17. As shown, the coupling device 1 is attached onto the robotically controlled endoscope system 11 at the robotically controlled endoscope system coupling device attachment region 13. As shown, the cable distal end 6 is secured with the coupling device 1 at the coupling device attachment region 2. As shown, the cable proximal end 4 is connected with generator 9 at the generator cable proximal end connection region 12. As shown, the cable 3 is engaged with the medical procedure device 10 at the cable distal end 6.

Such embodiments are not limited to a specific type of robotically controlled endoscope system 11. In some embodiments, the robotically controlled endoscope system 11 is selected from Monarch® (Auris™), Ion® (Intuitive®), and Galaxy System™ (Noah Medical™).

Such embodiments are not limited to a particular function for the robotically controlled endoscope system arms 14. In some embodiments, the robotically controlled endoscope system arms 14 are configured to assist in placement of a medical procedure device 10, manipulation of a medical procedure device 10, and/or providing a conduit for a medical procedure device 10 connected with the cable distal end 6 secured in a coupling device 1.

Still referring to FIG. 2, the coupling device 1 is not limited to a particular manner of attachment onto the robotically controlled endoscope system 11. In some embodiments, the coupling device 1 is configured to attach onto the robotically controlled endoscope system 11 at the robotically controlled endoscope system coupling device attachment region 13 such that the coupling device 1 is immobilized with respect to the robotically controlled endoscope system coupling device attachment region 13. The coupling device 1 is not limited to a particular manner of attachment onto the robotically controlled endoscope system coupling device attachment region 13. In some embodiments, the coupling device 1 attaches with the robotically controlled endoscope system coupling device attachment region 13 via one or more of: a magnet-based attachment, a shape-locking attachment, a suction-based attachment, a Velcro® attachment, and an adhesive attachment.

Still referring to FIG. 2, the coupling device 1 is not limited to a particular manner of securing the cable 3. In some embodiments, the coupling device 1 secures the cable distal end 6 such that cable distal end 6 is immobilized with respect to the coupling device 1.

Still referring to FIG. 2, the robotically controlled endoscope system 11 is not limited to a particular manner of connection with the cable 3. In some embodiments, the robotically controlled endoscope system cable proximal end connection region 12 connects with the cable proximal end 4 such the cable proximal end 4 is immobilized with respect to the robotically controlled endoscope system cable proximal end connection region 12.

Still referring to FIG. 2, the medical procedure device 10 is not limited to a particular manner of connection with the cable 3. In some embodiments, the medical procedure device 10 is releasably connected with the cable distal end 6.

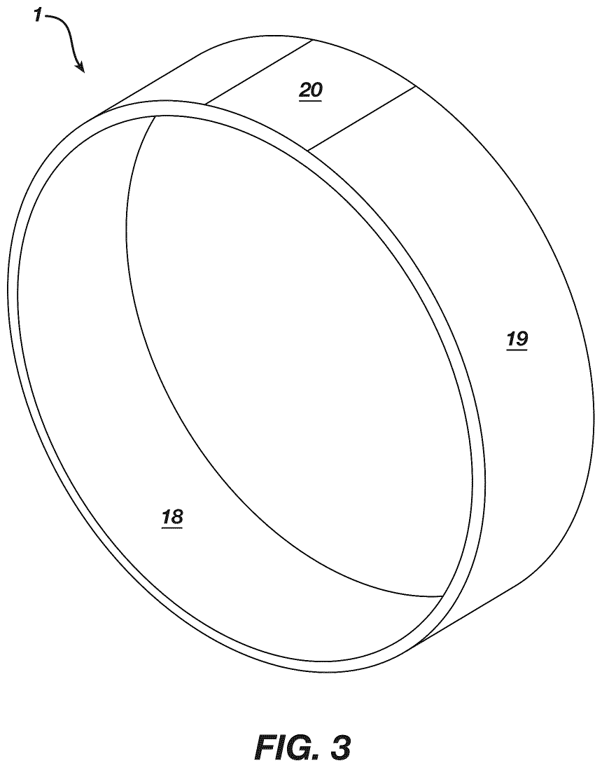

FIG. 3 shows an embodiment for a coupling device 1. Although the shape of the coupling device 1 shown in FIG. 3 is circular or oval, the coupling device 1 is not limited to particular shape or design. In some embodiments as shown in FIG. 3, the coupling device 1 has an open-hollow shape such that any item smaller in diameter than the diameter of the coupling device 1 can fit through the open-hollow shape. As shown, the coupling device has a coupling device interior surface 18, a coupling device exterior surface 19, and the coupling device exterior surface attachment region 20.

The coupling device is not limited to particular size. In some embodiments, the size of the coupling device is such that it can attach onto an endoscope system (e.g., robotically controlled endoscope system) without affecting the function of the endoscope system (e.g., robotically controlled endoscope system). In some embodiments, the size of the coupling device is such that it can secure a cable (e.g, cable distal end) without affecting the function of the cable (e.g., cable distal end). In some embodiments, the size of the coupling device is such that it can attach onto an endoscope system (e.g., robotically controlled endoscope system) without affecting the function of the endoscope system (e.g., robotically controlled endoscope system), and secure a cable (e.g, cable distal end) without affecting the function of the cable (e.g., cable distal end).

In some embodiments, the width of the coupling device is 36 inches or less (e.g., 36 inches or less, 35 inches or less, 34 inches or less, 33 inches or less, 32 inches or less, 31 inches or less, 30 inches or less, 29 inches or less, 28 inches or less, 27 inches or less, 26 inches or less, 25 inches or less, 24 inches or less, 23 inches or less, 22 inches or less, 21 inches or less, 20 inches or less, 19 inches or less, 18 inches or less, 17 inches or less, 16 inches or less, 15 inches or less, 14 inches or less, 13 inches or less, 12 inches or less, 11 inches or less, 10 inches or less, 9 inches or less, 8 inches or less, 7 inches or less, 6 inches or less, 5 inches or less, 4 inches or less, 3 inches or less, 2 inches or less, 1 inch or less).

In some embodiments, the height of the coupling device exterior surface and interior surface are the same. In some embodiments, the height of the coupling device exterior surface and coupling device interior surface are different. In some embodiments, the height of the coupling device exterior surface is larger than the height of the coupling device interior surface. In some embodiments, the height of the coupling device interior surface is larger than the height of the coupling device exterior surface. In some embodiments, the height of the coupling device interior surface and/or the height of the coupling device exterior surface is 36 inches or less (e.g., 36 inches or less, 35 inches or less, 34 inches or less, 33 inches or less, 32 inches or less, 31 inches or less, 30 inches or less, 29 inches or less, 28 inches or less, 27 inches or less, 26 inches or less, 25 inches or less, 24 inches or less, 23 inches or less, 22 inches or less, 21 inches or less, 20 inches or less, 19 inches or less, 18 inches or less, 17 inches or less, 16 inches or less, 15 inches or less, 14 inches or less, 13 inches or less, 12 inches or less, 11 inches or less, 10 inches or less, 9 inches or less, 8 inches or less, 7 inches or less, 6 inches or less, 5 inches or less, 4 inches or less, 3 inches or less, 2 inches or less, 1 inch or less).

In some embodiments, the coupling device is not limited to a particular composition. In some embodiments, the composition of the coupling device is plastic, metal, graphite, ceramic, and/or any combination thereof. In some embodiments, at least a portion of the coupling device is plastic (e.g., PEEK). In some embodiments, at least a portion of the coupling device comprises thermoplastic polymers that have an appropriate glass-transition temperature of approximately 15-25 degrees Celsius. In some embodiments, at least a portion of the coupling device comprises copolymers of lactic acid and caprolactone, wherein the thermoplastic polymer comprises a copolymer of L-lactide and caprolactone such as poly(L-lactide-co-caprolactone) with an L-lactide to caprolactone monomer ratio of 70:30 or less.

In some embodiments, the coupling device interior surface is configured to contact the robotically controlled endoscope system arm distal end such that it is capable of engaging the endoscope system coupling device attachment region (e.g., robotically controlled endoscope system coupling device attachment region).

In some embodiments, the coupling device attachment region and/or the coupling device exterior surface attachment region is configured to secure the cable distal end such that the cable distal end is immobilized with respect to the coupling device. In some embodiments, the coupling device is configured to attach onto one of the robotically controlled endoscope system arms such that the coupling device is immobilized with respect to the robotically controlled endoscope system arm. In some embodiments, the coupling device is configured to attach onto one of the robotically controlled endoscope system arm distal ends such that the coupling device is immobilized with respect to the robotically controlled endoscope system arm distal end.

The coupling device is not limited to a particular manner or mechanism for attachment onto the robotically controlled endoscope system arm distal end. In some embodiments, the coupling device is configured to attach onto one of the robotically controlled endoscope system arms via one or more of: a magnet-based attachment, a shape-locking attachment, a suction-based attachment, a Velcro® attachment, and an adhesive attachment.

In some embodiments, the endoscope system coupling device attachment region is not limited to a particular location. In some embodiments, the endoscope system coupling device attachment region is located along the robotically controlled endoscope system main body. In some embodiments, the endoscope system coupling device attachment region is located along the robotically controlled endoscope system distal end.

As noted, the coupling device is not limited to a particular shape. In some embodiments, the coupling device has an open-hollow shape. In some embodiments, the shape of the coupling device is such that it is able to match at least a portion of the shape of the robotically controlled endoscope system arm distal end (e.g., the coupling device is able to fit over and around the robotically controlled endoscope system arm distal end and attach onto the robotically controlled endoscope system arm distal end such that the coupling device is immobilized with respect to the robotically controlled endoscope system arm distal end). Indeed, in some embodiments, the coupling device is capable of attachment onto the robotically controlled endoscope system arm distal end by passing the robotically controlled endoscope system arm distal end through the open-hollow shape of the coupling device until the coupling device interior surface contacts the robotically controlled endoscope system arm distal end resulting in immobilization of the coupling device with respect to the robotically controlled endoscope system arm distal end.

In some embodiments, the coupling device is configured, upon being secured onto the endoscope system coupling device attachment region, to be tightened around the robotically controlled endoscope system arm distal end for purposes of further securing the coupling device with the relation to the robotically controlled endoscope system arm distal end. In some embodiments, the coupling device is configured, upon being secured onto the robotically controlled endoscope system arm distal end, to be loosened for purposes of releasing the coupling device from the robotically controlled endoscope arm system distal end.

The coupling device exterior surface attachment region is not limited to a particular manner of securing the cable distal end. In some embodiments, the coupling device exterior surface attachment region is configured to secure the cable distal end such that the cable distal end is immobilized with respect to the coupling device via one or more of: a magnet-based attachment, a shape-locking attachment, a suction-based attachment, a Velcro® attachment, and an adhesive attachment.

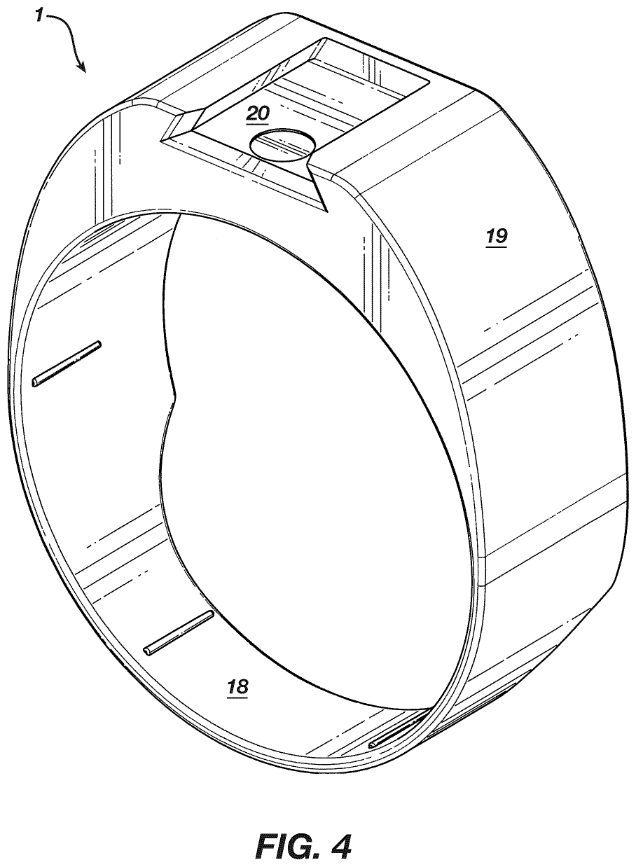

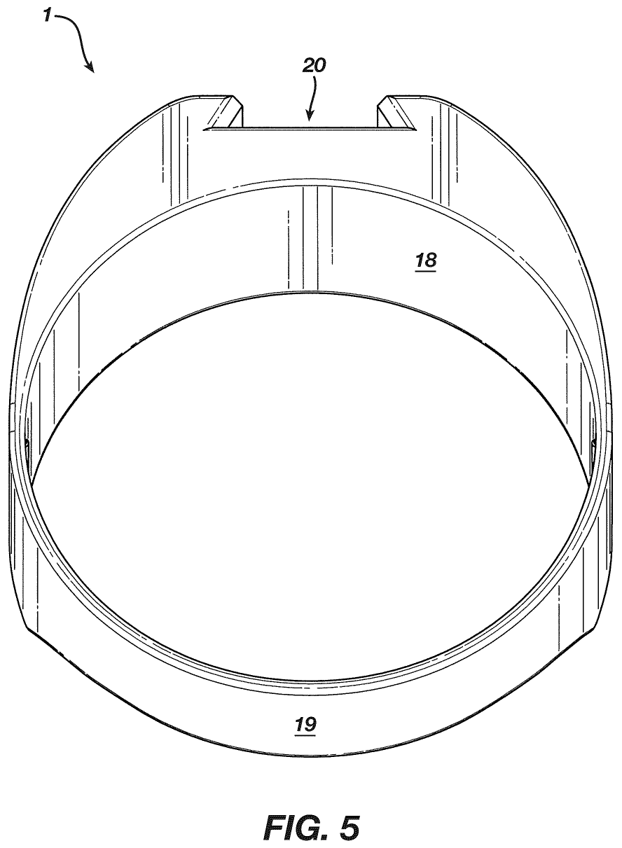

FIGS. 4-5 show different perspectives of coupling device 1 embodiments. As shown, the coupling device 1 has an open-hollow shape such that any item smaller in diameter than the diameter of the coupling device 1 can fit through the open-hollow shape. As shown, the coupling device 1 has a coupling device interior surface 18, a coupling device exterior surface 19, and the coupling device exterior surface attachment region 20.

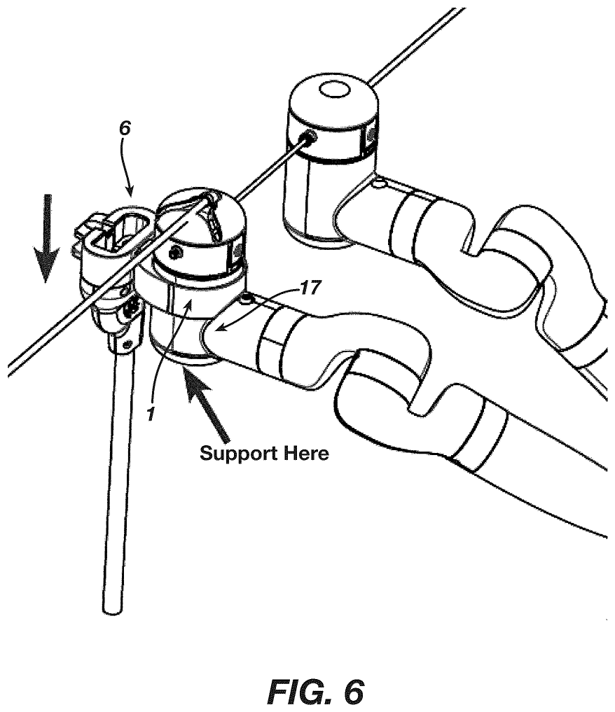

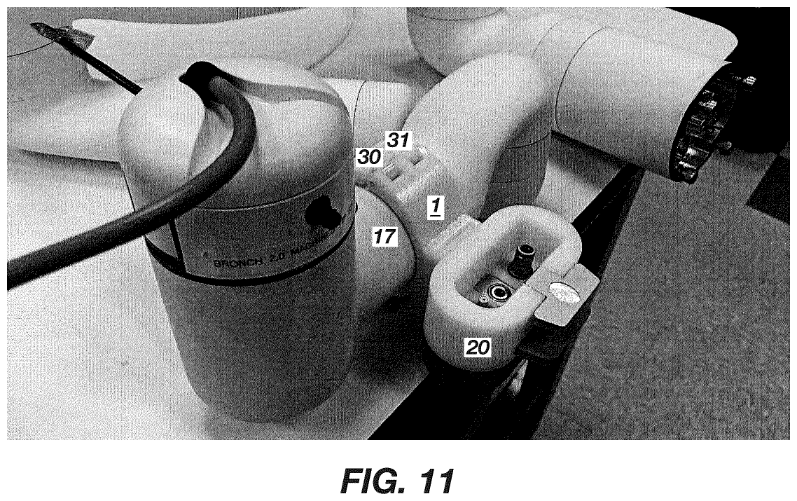

FIG. 6 shows a coupling device 1 attached onto a robotically controlled endoscope system arm distal end 17, and a cable distal end 6 secured with the coupling device 1. As shown in FIG. 11, the cable distal end 6 is not connected with a medical procedure device.

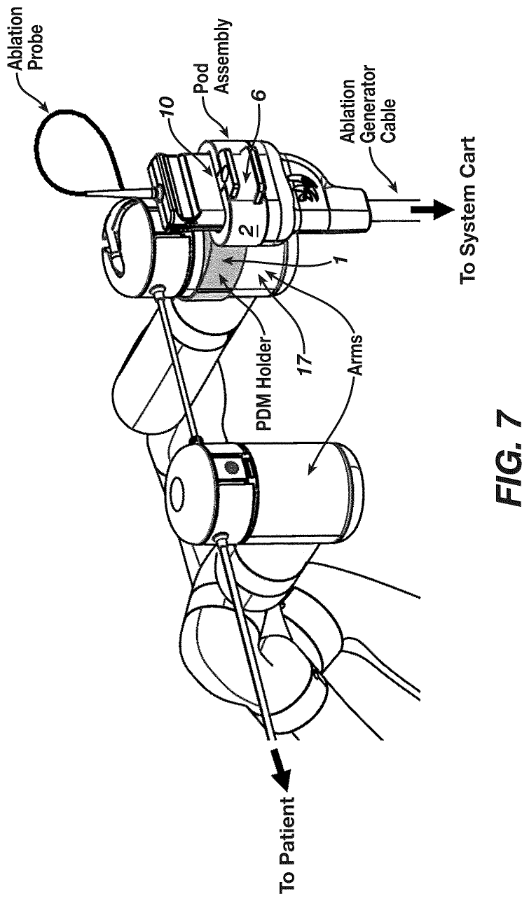

FIG. 7 shows a coupling device 1 attached onto a robotically controlled endoscope system arm distal end 17, and a cable distal end 6 secured with the coupling device 1. As shown in FIG. 11, the cable distal end 6 is connected with a medical procedure device 10.

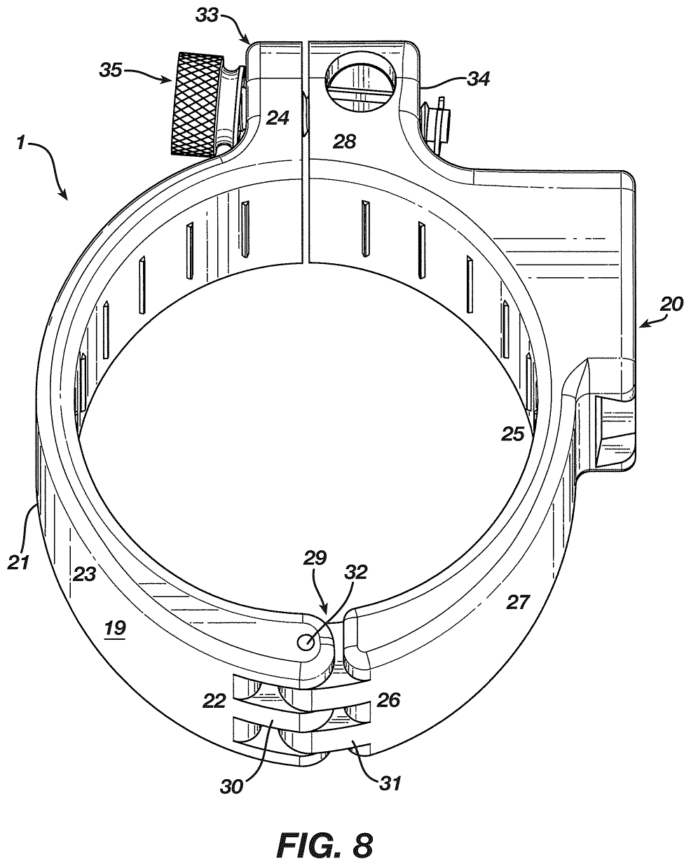

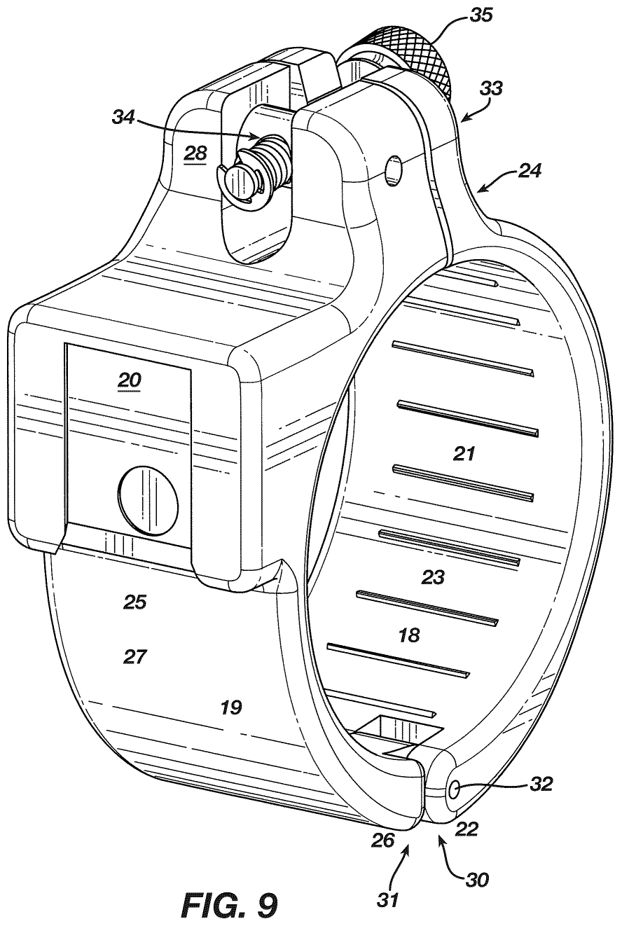

FIGS. 8-9 show an additional embodiment for the coupling device 1. As shown, the coupling device 1 is configured to be in an open-hollow closed loop shape or is configured to in a non-loop shape. As shown, the coupling device 1 has a coupling device first arm 21 having a coupling device first arm proximal end 22, a coupling device first arm main body 23, and a coupling device first arm distal end 24. As shown, the coupling device 1 has a coupling device second arm 25 having a coupling device second arm proximal end 26, a coupling device second arm main body 27, and a coupling device second arm distal end 28. As shown, the coupling device first arm 21 and coupling device second arm 25 are connected via a coupling device hinge 29. Indeed, as shown, the coupling device hinge 29 connects the coupling device first arm proximal end 22 and the coupling device second arm proximal end 26. As shown, the coupling device 1 further shows a coupling device interior surface 18, a coupling device exterior surface 19, and the coupling device exterior surface attachment region 20. As shown, the coupling device first arm distal end 24 is configured to lockingly connect with the coupling device second arm distal end 28 such that the coupling device first arm 21, the coupling device second arm 25, and the coupling device hinge 29 form an openable and closable loop.

Still referring to FIGS. 8-9, the coupling device 1 is not limited to a particular type or kind of coupling device hinge 29.

In some embodiments, the coupling device hinge 29 operates in such a manner without affecting the function of the endoscope system (e.g., robotically controlled endoscope system). In some embodiments, the coupling device hinge 29 operates in such a manner without affecting the function of the ability of the coupling device 1 to secure a cable (e.g., cable distal end) without affecting the function of the cable (e.g., cable distal end). In some embodiments, the coupling device hinge 29 operates in such a manner without affecting the function of the endoscope system (e.g., robotically controlled endoscope system), and secure a cable (e.g, cable distal end) without affecting the function of the cable (e.g., cable distal end).

In some embodiments, the coupling device 1 has a coupling device hinge 29 as shown in FIGS. 8-9. As shown, the coupling device first arm proximal end 22 has coupling device first arm proximal end knuckles 30, the coupling device second arm proximal end 25 has coupling device second arm proximal end knuckles 31, and a coupling device hinge pin 32. As shown, the coupling device first arm proximal end knuckles 30 and the coupling device second arm proximal end knuckles 31 are configured to intertwine and receive the coupling device hinge pin 32 thereby forming the coupling device hinge 29.

In some embodiments, the coupling device hinge 29 is configured such that a connected coupling device first arm 21 and coupling device second arm 25 (connected at the coupling device first arm proximal end 22 and coupling device second arm proximal end 26) are able to linearly rotate about the coupling device hinge 29 such that an expandable or retractable opening exists between the coupling device 1. Indeed, if the coupling device first arm distal end 24 and the coupling device second arm distal end 28 are connected, the coupling device first arm 21 and coupling device second arm 25 are unable to linearly rotate about the coupling device hinge 29 resulting in a closed-loop for the coupling device 1. Alternatively, if the coupling device first arm distal end 24 and the coupling device second arm distal end 28 are not connected, the coupling device hinge 29 permits the coupling device first arm 21 and coupling device second arm 25 to linearly rotate about the coupling device hinge 29 such that an expandable or retractable opening exists between the coupling device 1. In some embodiments, such an opening is adjustable and can be of a sufficient size such that at least a portion of the robotically controlled endoscope system arm distal end can be positioned within the opening.

In some embodiments, upon positioning of a robotically controlled endoscope arm system distal end within such an opening, the coupling device first arm 21 and coupling device second arm 25 can be linearly rotated about the coupling device hinge 29 resulting in connection of the coupling device first arm distal end 24 and the coupling device second arm distal end 28, and further resulting in the formation of a closed loop around the robotically controlled endoscope system arm distal end. In such embodiments, the coupling device 1 is thereby attached onto the robotically controlled endoscope system arm distal end. Further adjustment of the coupling device 1 (e.g., tightening of the formed closed-loop around the robotically controlled endoscope system arm distal end) can result in immobilization of the coupling device 1 with respect to the robotically controlled endoscope system arm distal end. Further adjustment of the coupling device 1 (e.g., loosening and subsequent tightening of the formed closed-loop around the robotically controlled endoscope system arm distal end) can result in refitting and subsequent immobilization of the coupling device 1 with respect to the robotically controlled endoscope system arm distal end. Further adjustment of the coupling device 1 (e.g., loosening of the formed closed-loop around the robotically controlled endoscope system arm distal end, and disconnection between the coupling device first arm distal end 24 and the coupling device second arm distal end 28) can result in removal of the coupling device 1 from the robotically controlled endoscope system arm distal end.

The coupling device 1 is not limited to a particular manner of tightening itself or loosening itself when the coupling device first arm distal end 24 and the coupling device second arm distal end 28 are connected. In some embodiments, as shown in FIGS. 8-9, the coupling device first arm distal end 24 has a coupling device first arm distal end opening 33, and the coupling device second arm distal end 28 has a coupling device second arm distal end opening 34. In some embodiments, the coupling device first arm distal end opening 33 and the coupling device second arm distal end opening 34 are each configured to linearly align for purposes of receiving a coupling device thumbscrew 35. In embodiments where the coupling device first arm distal end opening 33 and the coupling device second arm distal end opening 34 have received the coupling device thumbscrew 35, the coupling device thumbscrew 35 can be tightened (e.g., thereby tightening the coupling device 1 closed loop) or loosened (e.g., thereby loosening the coupling device 1 closed loop).

The coupling device exterior surface attachment region 20 is not limited to a particular positioning along the coupling device exterior surface 19. In some embodiments, as shown in FIGS. 8-9, the coupling device exterior surface attachment region 20 is positioned within along either the exterior surface of the coupling device first arm exterior surface main body 23 or the exterior surface of the coupling device second arm exterior surface main body 27.

The coupling device exterior surface attachment region is not limited to a particular manner of securing the cable distal end. In some embodiments, the coupling device exterior surface attachment region is configured to secure the cable distal end such that the cable distal end is immobilized with respect to the coupling device via one or more of: a magnet-based attachment, a shape-locking attachment, a suction-based attachment, a Velcro® attachment, and an adhesive attachment.

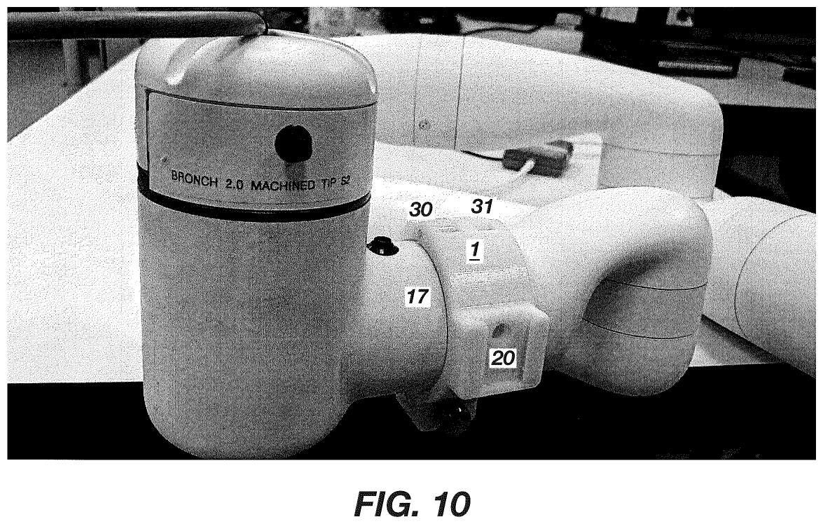

FIGS. 10 and 11 show a coupling device 1 attached onto a robotically controlled endoscope system arm distal end 17. As shown, the coupling device first arm proximal end knuckles 30 and the coupling device second arm proximal end knuckles 31 are intertwined and connected via the coupling device hinge pin 32 thereby forming the coupling device hinge 29. As shown, a cable distal end is secured with the coupling device exterior surface attachment region 20.

In some embodiments, the present invention provides systems for medical procedures providing a coupling device as described herein, an endoscope system (e.g., a robotically controlled endoscope system as described herein), a cable as described herein, and a medical procedure device (e.g., a microwave ablation device) (e.g., any type or kind of ablation device) (e.g., any energy delivery device described herein).

The coupling devices described herein are not limited to particular uses. Indeed, such coupling devices and systems of the present invention are designed for use in any setting wherein use of an endoscope system (e.g., robotically controlled endoscope system) is applicable. For example, the coupling devices and systems find use for open surgery, percutaneous, intravascular, intracardiac, intraluminal, endoscopic, laparoscopic, or surgical delivery of energy. Such uses include any and all medical, veterinary, and research applications. In addition, the systems and coupling devices of the present invention may be used in agricultural settings, manufacturing settings, mechanical settings, or any other application where energy is to be delivered.

The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the systems including the flexible sheath described herein are configured for movement and positioning, with minimal damage to the tissue or organism, at any desired location, including but not limited to, the lungs, brain, neck, chest, abdomen, and pelvis. In some embodiments, the systems are configured for guided delivery, for example, by computerized tomography, ultrasound, magnetic resonance imaging, fluoroscopy, and the like. Indeed, in some embodiments, all inserted components of such a system are configured for movement along a narrow and circuitous path through a subject (e.g. through a branched structure, through the bronchial tree, etc.).

In certain embodiments, the present invention provides methods of treating a tissue region, comprising providing a tissue region and a system described herein (e.g., a coupling device as described herein, a robotically controlled endoscope system as described herein, and a medical procedure device (e.g., a microwave ablation device) (e.g., any type or kind of ablation device) (e.g., any energy delivery device described herein)); securing the cable proximal end with the robotically controlled endoscope system; securing the coupling device onto one of the robotically controlled endoscope system arm distal ends such that the coupling device is immobilized with respect to the robotically controlled endoscope system distal arm; securing the cable distal end with the coupling device attachment region such that the cable distal end is immobilized with respect to the coupling device; connecting the medical procedure device with the cable distal end; contacting a subject (e.g., human subject) with the medical procedure device connected with the cable distal end; and administering to the subject a medical procedure with the medical procedure device connected with the cable distal end. In some embodiments, the medical procedure is delivery of microwave ablation therapy to a specific tissue region. In some embodiments, the tissue region is a tumor. In some embodiments, administration of the medical procedure results in, for example, the ablation of the tissue region and/or thrombosis of a blood vessel, and/or electroporation of a tissue region. In some embodiments, the tissue region comprises one or more of the lung, heart, liver, genitalia, stomach, lung, large intestine, small intestine, brain, neck, bone, kidney, muscle, tendon, blood vessel, prostate, bladder, and spinal cord.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-45. (canceled)

46. A coupling device for a robotic system, the coupling device comprising:
a handle with a proximal end, a distal end, a first surface, and second surface, wherein the first surface is opposed the second surface;
the distal end defining a first radius with a first axis;
an aperture through the handle, the aperture defining a second radius about the first axis, the second radius being smaller than the first radius; and
an attachment portion on the proximal end, the attachment portion further comprising a recess in the handle extending from the first surface and terminating before the second surface.

47. The coupling device of claim 46, wherein the recess defines a dovetail shape.

48. The coupling device of claim 46, wherein the handle further comprises one or more protrusions that extend into the aperture.

49. The coupling device of claim 48, wherein the protrusions are spaced apart about the circumference of the aperture.

50. The coupling device of claim 49, wherein the attachment portion further comprises a magnet.

51. The coupling device of claim 50, wherein the handle has a third surface and fourth surface, the third surface defined by the aperture and the fourth surface defined by a radial offset from the third surface.

52. The coupling device of claim 51, wherein the radial offset is greater on the proximal end than the distal end.

53. The coupling device of claim 52, wherein the first surface and second surface define a handle height and the handle height is greater on the proximal end than the distal end.

54. A robotic surgical system, comprising:
at least one surgical arm with a first end and second end;
an instrument interface at the first end of the surgical arm;
a first instrument mounted to the first instrument interface; and
a coupling device positioned between the first end and the second end of the at least one surgical arm, wherein the coupling device further comprises:
a handle with a proximal end, a distal end, a first surface, and second surface, where in the first surface is opposed the second surface;
the distal end defining a first radius with a first axis;
an aperture through the handle, the aperture defining a second radius about the first axis, the second radius being smaller than the first radius; and
an attachment portion on the proximal end, the attachment portion further comprising a recess into the handle extending from the first surface and terminating before the second surface.

55. The robotic surgical system of claim 54 further comprising, a second instrument mounted to the coupling device.

56. The robotic surgical system of claim 55, wherein the second surgical instrument is configured for insertion and withdrawal through a working channel on the first surgical instrument.

57. The robotic surgical system of claim 56, wherein the recess defines a dovetail shape.

58. The robotic surgical system of claim 56, wherein the handle further comprises one or more protrusions that extend into the aperture.

59. The robotic surgical system of claim 58, wherein the protrusions are spaced apart about the circumference of the aperture.

60. The robotic surgical system of claim 59, wherein the attachment portion further comprises a magnet.

61. The robotic surgical system of claim 60, wherein the handle has a third surface and fourth surface, the third surface defined by the aperture and the fourth surface defined by a radial offset from the third surface.

62. The robotic surgical system of claim 61, wherein the radial offset is greater on the proximal end than the distal end.

63. The robotic surgical system of claim 62, wherein the first surface and second surface define a handle height and the handle height is greater on the proximal end than the distal end.

64. A method of coupling a surgical instrument to a robotic surgical system comprising:
sliding a coupling device over a first instrument interface located at a first end of at least one surgical arm to a position between the first end and a second end of the at least one surgical arm;
mounting a first instrument to the first instrument interface; and
mounting a second instrument to the coupling device;

65. The method of claim 64, further comprising of inserting the second instrument into a working channel of the first instrument.
